(19) United States
(12) Patent Application Publication
(10) Pub. No.: US 2026/0183384 A1
(43) Pub. Date: Jul. 2, 2026

(54) ADJUVANT AND TRANSMUCOSAL VACCINE

(71) Applicant: JUNTENDO EDUCATIONAL FOUNDATION, Tokyo (JP)

(72) Inventors: Hiraku SASAKI, Bunkyo-ku (JP); Yoshio SUZUKI, Bunkyo-ku (JP); Hiroki ISHIKAWA, Shinagawa-ku (JP)

(73) Assignee: JUNTENDO EDUCATIONAL FOUNDATION, Tokyo (JP)

(21) Appl. No.: 19/113,080

(22) PCT Filed: Sep. 29, 2023
(86) PCT No.: PCT/JP2023/035586

§ 371 Date: Mar. 19, 2025

(30) Foreign Application Priority Data
Sep. 30, 2022 (JP) 2022-158607

(51) Int. Cl. A61K 39/215; A61K 9/00; A61K 39/00; A61K 39/385; A61K 39/39; A61P 31/14

(52) U.S. Cl. CPC A61K 39/215; A61K 9/0043; A61K 39/385; A61K 39/39; A61P 31/14; A61K 2039/543; A61K 2039/55544; A61K 2039/575; A61K 2039/6037; C12N 2770/20034

(57) ABSTRACT

An adjuvant and a transmucosal vaccine may have a high ability of inducing, through transmucosal administration, secretory IgA in addition to serum IgA and IgG. Such an adjuvant may contain, as an active ingredient, one or more polypeptides selected from (1) a polypeptide having one or two amino acid sequences shown in at least SEQ ID NO: 1, and (2) a polypeptide having 93% or more identity to the amino acid sequence.

### TECHNICAL FIELD

The present invention relates to an adjuvant and a transmucosal vaccine.

### BACKGROUND ART

Since novel coronavirus (SARS-CoV-2) and influenza virus usually invade through the upper respiratory tract, use of a transmucosal vaccine not only can induce IgG in the serum but also can induce secretory IgA in the upper respiratory mucosa including the nasal cavity, and thus, the invasion of the virus itself can be inhibited.

On the other hand, vaccination by intramuscular injection or the like produces IgG in the serum, and the IgG protects against the virus through the blood. Accordingly, it is considered that such a vaccine has a strong effect of preventing aggravation but has a weak ability of preventing infection itself with the virus. Besides, a side effect of the intramuscular injection has become an issue.

From this point of view, development of transmucosal vaccines such as a nasal vaccine has been desired, and a live attenuated influenza vaccine (product name: FluMist) in a form to be sprayed into the nasal cavity has been developed (Non Patent Literature 1).

Also as for SARS-CoV-2, transmucosal vaccines have been developed, but none has been commercialized yet.

### CITATION LIST

### Non Patent Literature

Non Patent Literature 1: http://www.slclinic.com/aboutus/ichikawaclinic/flumist/Non
Patent Literature 2: Vaccine, 30 Sep. 2013, 31 (48): 5729-5735

### SUMMARY OF INVENTION

### Technical Problem

As an adjuvant of an injectable vaccine, an aluminum salt, adjuvant MF59, and the like are used. Regarding an adjuvant of a transmucosal vaccine, there have been research reports, but none has been put to practical use yet.

An object of the present invention is to provide an adjuvant and a transmucosal vaccine having a high ability of inducing secretory IgA in addition to serum IgA and IgG through transmucosal administration.

### Solution to Problem

The present inventors have found that when a peptide obtained by eliminating cytotoxicity from PnxIIIA protein of a rodent opportunistic pathogen, Pasteurella pneumotropica (MP3 (100 kDa)) is nasally inoculated, antibodies (IgA and IgG) against MP3 are induced singly without needing an adjuvant (Non Patent Literature 2). The MP3 is, however, a large polypeptide having a molecular weight of 100 kDa, and functions as an antigen by itself, and hence cannot be used as an adjuvant.

Therefore, the present inventors have further removed, from the MP3, portions predicted to be cytotoxic and immunogenic for redesigning it to be easily expressed in E. coli, and thus, have found polypeptides containing at least one or two specific 61-amino acid residues. Besides, the following has been found: When these polypeptides are bound to an antigen such as a viral antigen, and the resultants are transmucosally administered, not an antibody against MP3 but high IgG and IgA antibody titers against the antigen bound thereto are obtained, and these polypeptides have very low cytotoxicity. Thus, the present invention has been accomplished.

Specifically, the present invention provides the following inventions [1] to [15]:

[1] An adjuvant comprising, as an active ingredient, one or more polypeptides selected from the group consisting of (1) a polypeptide having one or two amino acid sequences shown in at least SEQ ID NO: 1, and (2) a polypeptide having 93% or more identity to the amino acid sequence.

[2] An adjuvant comprising, as an active ingredient, one or more polypeptides selected from the group consisting of (1) a polypeptide having one or two amino acid sequences shown in at least SEQ ID NO: 1, and (2) a polypeptide having 95% or more identity to the amino acid sequence.

[3] The adjuvant according to [1] or [2], wherein the amino acid sequence shown in SEQ ID NO: 1 is an amino acid sequence shown in SEQ ID NO: 2 or 3.

[4] An adjuvant comprising, as an active ingredient, a polypeptide having one or two amino acid sequences shown in at least SEQ ID NO: 2 or 3.

[5] The adjuvant according to [4], wherein the polypeptide having one amino acid sequence shown in at least SEQ ID NO: 2 is a polypeptide having an amino acid sequence shown in SEQ ID NO: 4.

[6] The adjuvant according to [5], wherein the polypeptide having the amino acid sequence shown in SEQ ID NO: 4 is a polypeptide having an amino acid sequence selected from the group consisting of SEQ ID NOS: 5 to 9.

[7] The adjuvant according to [3], wherein the polypeptide having two amino acid sequences shown in at least SEQ ID NO: 1 is a polypeptide having an amino acid sequence shown in SEQ ID NO: 10.

[8] The adjuvant according to [7], wherein the polypeptide having the amino acid sequence shown in SEQ ID NO: 10 is a polypeptide having an amino acid sequence selected from the group consisting of SEQ ID NOS: 11 to 15.

[9] A polypeptide comprising, as an active ingredient, a conjugate of the adjuvant according to any one of [1] to [8] with an antigen, for producing an antibody against the antigen.

[10] A transmucosal vaccine comprising, as an active ingredient, a conjugate of the adjuvant according to any one of [1] to [8] with a pathogen, a substance produced by the pathogen, or a part of the pathogen or substance.

[11] The transmucosal vaccine according to [10], wherein the pathogen, the substance produced by the pathogen, or the part of the pathogen or substance is a pathogen selected from the group consisting of influenza virus, coronavirus, human papillomavirus, measles virus, tetanus toxin, and pertussis toxin, or a substance produced by the pathogen, or a part of the pathogen or substance.

[12] Use of a conjugate of the adjuvant according to any one of [1] to [8] with a pathogen, a substance produced by the pathogen, or a part of the pathogen or substance for producing a transmucosal vaccine.

[13] A conjugate of the adjuvant according to any one of [1] to [8] with a pathogen, a substance produced by the pathogen, or a part of the pathogen or substance for use in a transmucosal vaccine therapy.

[14] A transmucosal vaccine therapy comprising transmucosally administering a conjugate of the adjuvant according to any one of [1] to [5] with a pathogen, a substance produced by the pathogen, or a part of the pathogen or substance.

[15] An antigen for producing a transmucosal vaccine against coronavirus or SARS-CoV-2 comprising a polypeptide having an amino acid sequence shown in SEQ ID NO: 16 (OC43ag), or a polypeptide having an amino acid sequence having 95% or more identity to the amino acid sequence.

### Advantageous Effects of Invention

When a conjugate of the adjuvant of the present invention with an antigen is transmucosally administered, not only IgA and IgG can be induced in the serum with a high antibody titer but also a secretory IgA against the antigen can be induced in the mucosa with a high antibody titer, and therefore, the adjuvant of the present invention can cause production of a specific antibody against a polypeptide having low immunogenicity.

When a conjugate of the adjuvant of the present invention with a pathogen, a substance produced by the pathogen, or a part of the pathogen or substance is transmucosally administered, secretory IgA can be induced in the mucosa with a high antibody titer. Therefore, invasion of the pathogen having the antigen through the mucosa can be prevented, and IgA and IgG can be induced also in the serum with a high antibody titer, and thus, an infectious disease caused by the pathogen which has entered the body, or a substance produced by the pathogen can be prevented.

Besides, a transmucosal vaccine of the present invention can induce IgA and IgG in the serum with a high antibody titer through transmucosal administration such as nasal administration, and in addition, can induce secretory IgA in the mucosa with a high antibody titer, and therefore, it is possible to also reduce a side reaction such as attenuated live vaccines and nucleic acid vaccines and pains caused in inoculation of injectable vaccines.

### BRIEF DESCRIPTION OF DRAWINGS

FIG. 1 illustrates a flow of an adjuvant of the present invention (NAIS291-1) and a part of spike protein of human coronavirus OC43 (OC43 vaccine antigen: OC43ag).

FIG. 2 illustrates an anti-HCoV-OC43 spike protein antibody titer in serum obtained when a transmucosal vaccine of the present invention was nasally administered.

FIG. 3 illustrates cytotoxicity of the adjuvant of the present invention (NAIS291-1) against L929 cells.

FIG. 4 illustrates anti-thioredoxin antibody titer and anti-NAIS antibody titer obtained when conjugates of adjuvants of the present invention (NAIS61-2, NAIS230-1, and NAIS291-1) with thioredoxin (antigen) were nasally administered.

FIG. 5 illustrates anti-HA antibody titers obtained when conjugates of the adjuvants of the present invention (NAIS61-2, and NAIS230-1) with hemagglutinin (HA) were nasally administered.

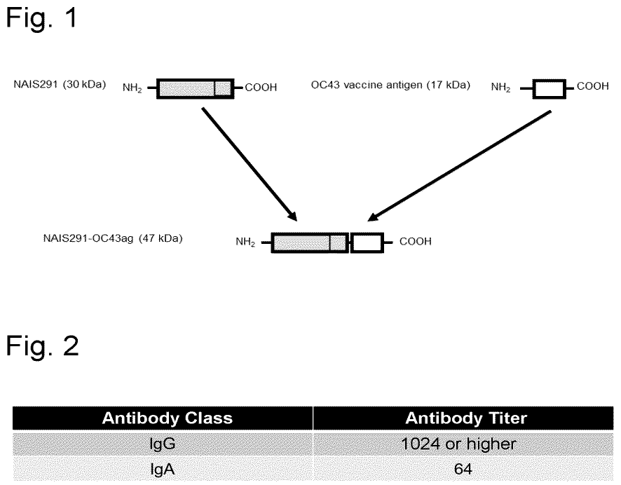

### DESCRIPTION OF EMBODIMENTS

Terms used herein are used in the meanings usually employed in this technical field unless otherwise stated. The present invention will now be described in more detail.

The term “adjuvant” means an auxiliary agent relative to a main agent, is also designated as an immunogenicity enhancer in the field of immunology, and refers to a substance which is injected together with an antigen for enhancing the immunogenicity. In the field of preventive medicine, an adjuvant refers to a substance used together with a vaccine for enhancing the effect. The adjuvant of the present invention has these meanings of both the fields of immunology and the preventive medicine.

The term “antigen” refers to a substance which enters a living body to produce an antibody, and binds only to the antibody to cause a reaction, and bacterial toxins, bacterial cell components, viruses, and a large number of heterologous proteins correspond to the antigen.

The term “pathogen” refers to organisms or the like such as protozoans, bacterium, and viruses which parasitize a living body to cause a disease, and particularly those having pathogenicity. These pathogens have immunogenicity, and hence can be regarded as a type of antigen. Examples of a substance produced by a pathogen include bacterial toxins. Examples of a part of the pathogen include a bacterial cell component, a membrane protein of a virus, a spike protein produced by a virus, and a part of these.

The term “antibody” generally refers to a protein which is produced in a living body through stimulation with an antigen, and is specifically reacted with this antigen. Specifically, the antibody is a glycoprotein produced by a B cell, that is, a type of lymphocytes which are one of subtypes of leukocytes, and is designated also as an immunoglobulin. Immunoglobulins include IgG, IgE, IgA, IgM, and IgD.

The term “vaccine” refers to a pharmaceutical used for preventing an infectious disease by acquiring immunity to the infectious disease. Specifically, the vaccine refers to a pharmaceutical which accelerates production of an antibody against a pathogen in a living body to acquire immunity to an infectious disease by administration of a detoxified or attenuated antigen itself produced from the pathogen or a chemically synthesized gene sequence of an mRNA or DNA designed based on the pathogen (genetic vaccine), or a protein highly expressed by genetic engineering (recombinant vaccine).

The term “mucosa” refers to a soft tissue present on the inner surface of a hollow organ such as a digestive organ, a respiratory organ, and a urogenital organ. Accordingly, transmucosal administration refers to administration directly to such mucosa. There is mucosa in a nasal cavity or an oral cavity, and direct administration to such mucosa is preferred in the present invention.

The term “amino acid sequence identity” refers to a ratio (%), in two amino acid sequences aligned, of the number of positions having the same amino acid residues in the both sequences to the number of full-length amino acid residues. Specifically, the amino acid sequence identity is calculated by, for example, Lipman-Pearson method (Science, 227, 1435 (1985)), and can be calculated by analysis with a homology analysis (Search homology) program of genetic information processing software, Genetyx-Win (Ver. 5.1.1; Software Development Co., Ltd.) with Unit size to compare (ktup) set to 2.

One embodiment of the present invention relates to an adjuvant containing, as an active ingredient, one or more polypeptides selected from the group consisting of (1) a polypeptide having one or two amino acid sequences shown in at least SEQ ID NO: 1, and (2) a polypeptide having 93% or more identity to the amino acid sequence.

The amino acid sequence shown in SEQ ID NO: 1 is shown in Table 1.

TABLE 1
TLNX¹DGTLNX²PAGVKEFTITTPVKADNTTEGEEKGKFT
VGGVEX³NEVTVNDTSX⁴XXXX⁸PX⁹X¹⁰
(SEQ ID NO: 1)

In the amino acid sequence, X¹ represents S or Q, X² represents I or V, X³ represents G or S, X⁴ represents A or K, X⁵ represents T or Q, X⁶ represents P or D, X⁷ represents A or E, X⁸ represents D or Q, X⁹ represents A or K, and X¹⁰ represents D or I.

The amino acid sequence shown in SEQ ID NO: 1 has 61 amino acid residues. Each of ten amino acid residues (X¹ to X¹⁰) of the amino acid sequence is either of the two amino acids as described above. It is not known that the polypeptide having the 61 amino acid residues functions as an adjuvant. A polypeptide having the amino acid sequence shown in SEQ ID NO: 1 is referred to as Nasal Antigen-Inducible Sequence (NAIS) 61 in some cases.

The polypeptide used in the present invention includes the polypeptide (2) having 93% or more identity to the amino acid sequence of SEQ ID NO: 1. The polypeptide is preferably a polypeptide having 95% or more identity to the amino acid sequence of SEQ ID NO: 1, more preferably a polypeptide having 97% or more identity to the amino acid sequence of SEQ ID NO: 1, further more preferably a polypeptide having 98% or more identity to the amino acid sequence of SEQ ID NO: 1, and even more preferably a polypeptide having 99% or more identity to the amino acid sequence of SEQ ID NO: 1.

A specific example of the 61 amino acid polypeptide having the amino acid sequence shown in SEQ ID NO: 1 may be a polypeptide having any one of the two amino acid residues indicated by X¹ to X¹⁰ described above, and a particularly preferable one is a polypeptide having an amino acid sequence shown in the following SEQ ID NO: 2 or 3:

TABLE 2
TLNSDGTLNIPAGVKEFTITTPVKADNTTEGEEKGKFTVG
GVEGNEVTVNDTSATPADPAD
(SEQ ID NO: 2, NAIS61-1)

TABLE 3
TLNSDGTLNIPAGVKEFTITTPVKADNTTEGEEKGKFTVG
GVEGNEVTVNDTSKQDEQPKI
(SEQ ID NO: 3, NAIS61-2)

The polypeptide used in the adjuvant of the present invention may contain one or two polypeptides having an amino acid sequence shown in at least SEQ ID NO: 1, or having 93% or more identity to the amino acid sequence, and a polypeptide having one or two amino acid sequences shown in SEQ ID NO: 1 is more preferred.

As an example of a polypeptide having one amino acid sequence shown in SEQ ID NO: 1, a polypeptide having an amino acid sequence shown in the following SEQ ID NO: 4 is more preferred from the viewpoint of obtaining a higher adjuvant effect. In the amino acid sequence of SEQ ID NO: 4, 61 amino acids from the N terminal preferably correspond to NAIS61-1.

TABLE 4
TLNX¹DGTLNX²PAGVKEFTITTPVKADNTTEGEEKGKFT
VGGVEX³NEVTVNDTSATPADPADKPTITSPDNDGKVTVE
PGADNNKVEX¹¹TFKDEDGX¹²DKTVVAEKGX¹³DGNWTX¹⁴
TX¹⁵DDGTGATIEGGKVVIPADKVKDGEPVNAKGTNDAGN
SENADAVNAGTDPKDAGVX¹⁶NTNGDGVVSAPVSADEGT
X¹⁷IVTTVKLNNNNGNEKLPFSLPSGTGNGQLTADDFEQ
PTFSNGV
(SEQ ID NO: 4)

In the amino acid sequence, X¹ represents S or Q, X² represents I or V, X³ represents G or S, X¹¹ represents V or A, X¹² represents N or K, X¹³ represents Q or T, X¹⁴ represents I or V, X¹⁵ represents N or Q, X¹⁶ represents D or N, and X¹⁷ represents S or N.

The amino acid sequence shown in SEQ ID NO: 4 contains 230 amino acid residues. Each of ten amino acid residues (X¹ to X¹⁷) of this amino acid sequence is either of the two amino acids as described above. It is not known that the polypeptide having the 230 amino acid residues functions as an adjuvant. A polypeptide having the amino acid sequence shown in SEQ ID NO: 4 is referred to as NAIS230 in some cases.

Specific examples of the polypeptide having the amino acid residues shown in SEQ ID NO: 4 include polypeptides having amino acid sequences shown in the following SEQ ID NO: 5 to 9:

TABLE 5
TLNSDGTLNIPAGVKEFTITTPVKADNTTEGEEKGKFTVGGVEGN
EVTVNDTSATPADPADKPTITSPDNDGKVTVEPGADNNKVEVTFK
DEDGNDKTVVAEKGQDGNWTIINDDGTGATIEGGKVVIPADKVKD
GEPVNAKGTNDAGNSENADAVNAGTDPKDAGVNNTNGDGVVSAPV
SADEGTNIVTTVKLNNNNGNEKLPFSLPSGTGNGQLTADDFEQPT
FSNGV
(SEQ ID NO: 5)

TABLE 6
TLNSDGTLNIPAGVKEFTITTPVKADNTTEGEEKGKFTVGGVEGN
EVTVNDTSATPADPADKPTITSPDNDGKVTVEPGADNNKVEATFK
DEDGNDKTVVAEKGQDGNWTITNDDGTGATIEGGKVVIPADKVKD
GEPVNAKGTNDAGNSENADAVNAGTDPKDAGVDNTNGDGVVSAPV
SADEGTSIVTTVKLNNNNGNEKLPFSLPSGTGNGQLTADDFEQPT
FSNGV
(SEQ ID NO: 6)

TABLE 7
TLNSDGTLNIPAGVKEFTITTPVKADNTTEGEEKGKFTVGGVEGN
EVTVNDTSATPADPADKPTITSPDNDGKVTVEPGADNNKVEVTFK
DEDGNDKTVVAEKGQDGNWTIINDDGTGATIEGGKVVIPADKVKD
GEPVNAKGTNDAGNSENADAVNAGTDPKDAGVDNTNGDGVVSAPV
SADEGTNIVTTVKLNNNNGNEKLPFSLPSGTGNGQLTADDFEQPT
FSNGV
(SEQ ID NO: 7)

TABLE 8
TLNSDGTLNIPAGVKEFTITTPVKADNTTEGEEKGKFTVGGVEGN
EVTVNDTSATPADPADKPTITSPDNDGKVTVEPGADNNKVEVTFK
DEDGNDKTVVAEKGQDGNWTIINDDGTGATIEGGKVVIPADKVKD
GEPVNAKGTNDAGNSENADAVNAGTDPKDAGVDNTNGDGVVSAPV
SADEGTSIVTTVKLNNNNGNEKLPFSLPSGTGNGQLTADDFEQPT
FSNGV
(SEQ ID NO: 8)

TABLE 9
TLNQDGTLNVPAGVKEFTITTPVKADNTTEGEEKGKFTVGGVESN
EVTVNDTSATPADPADKPTITSPDNDGKVTVEPGADNNKVEVTFK
DEDGKDKTVVAEKGTDGNWTVTQDDGTGATIEGGKVVIPADKVKD
GEPVNAKGTNDAGNSENADAVNAGTDPKDAGVDNTNGDGVVSAPV
SADEGTNIVTTVKLNNNNGNEKLLESLPNGTGNGQLTADDFEQPT
FSNGV
(SEQ ID NO: 9)

Among these, an example of a polypeptide having two amino acid sequences shown in SEQ ID NO: 1 is, from the viewpoint of obtaining a higher adjuvant effect, more preferably a polypeptide having an amino acid sequence shown in the following SEQ ID NO: 10. In the amino acid sequence of SEQ ID NO: 10, 61 amino acids from the N terminal preferably correspond to NAIS61-1.

TABLE 10
TLNX¹DGTLNX²PAGVKEFTITTPVKADNTTEGEEKGKFT
VGGVEX³NEVTVNDTSATPADPADKPTITSPDNDGKVTVE
PGADNNKVEX¹¹TFKDEDGX¹²DKTVVAEKGX¹³DGNWTX¹⁴
TX¹⁵DDGTGATIEGGKVVIPADKVKDGEPVNAKGTNDAGN
SENADAVNAGTDPKDAGVX¹⁶NTNGDGVVSAPVSADEGTX¹⁷
IVTTVKLNNNNGNEKLPFSLPSGTGNGQLTADDFEQPTF
SNGVTLNSDGTLNIPAGVKEFTITTPVKADNTTEGEEKG
KFTVGGVEGNEVTVNDTSKQDEQPKI
(SEQ ID NO: 10)

In the amino acid sequence, X¹ represents S or Q, X² represents I or V, X³ represents G or S, X¹¹ represents V or A, X¹² represents N or K, X¹³ represents Q or T, X¹⁴ represents I or V, X¹⁵ represents N or Q, X¹⁶ represents D or N, and X¹⁷ represents S or N.

The amino acid sequence shown in SEQ ID NO: 10 has 291 amino acid residues. Each of ten amino acid residues (X¹ to X¹⁷) of this amino acid sequence is either of the two amino acids described above. The 61 amino acids on the N terminal side in the amino acid sequence shown in SEQ ID NO: 10 preferably correspond to an amino acid sequence shown in SEQ ID NO: 2 (NAIS61-1), and 61 amino acids on the C terminal side correspond to an amino acid sequence shown in SEQ ID NO: 3 (NAIS61-2).

It is not known that the polypeptide having the 291 amino acid residues functions as an adjuvant. A polypeptide having the amino acid sequence shown in SEQ ID NO: 10 is referred to as NAIS291 in some cases.

Specific preferable examples of the NAIS291 used in the present invention include polypeptides having amino acid sequences selected from the group consisting of the following SEQ ID NOS: 11 to 15. The amino acid sequence shown in SEQ ID NO: 11 is a polypeptide in which NAIS61-2 of SEQ ID NO: 3 is bound to the C terminal of the amino acid sequence of SEQ ID NO: 5. A polypeptide having an amino acid sequence shown in SEQ ID NO: 11 is referred to as NAIS291-1 in some cases.

Similarly, a polypeptide in which NAIS61-2 of SEQ ID NO: 3 is bound to the C terminal of an amino acid sequence of SEQ ID NO: 6 (SEQ ID NO: 12), a polypeptide in which NAIS61-2 of SEQ ID NO: 3 is bound to the C terminal of an amino acid sequence of SEQ ID NO: 7 (SEQ ID NO: 13), a polypeptide in which NAIS61-2 of SEQ ID NO: 3 is bound to the C terminal of an amino acid sequence of SEQ ID NO: 8 (SEQ ID NO: 14), and a polypeptide in which NAIS61-2 of SEQ ID NO: 3 is bound to the C terminal of an amino acid sequence of SEQ ID NO: 9 (SEQ ID NO: 15) are as follows:

TABLE 11
TLNSDGTLNIPAGVKEFTITTPVKADNTTEGEEKGKFTVGGVEGN
EVTVNDTSATPADPADKPTITSPDNDGKVTVEPGADNNKVEVTFK
DEDGNDKTVVAEKGQDGNWTIINDDGTGATIEGGKVVIPADKVKD
GEPVNAKGTNDAGNSENADAVNAGTDPKDAGVNNTNGDGVVSAPV
SADEGTNIVTTVKLNNNNGNEKLPFSLPSGTGNGQLTADDFEQPT
FSNGVTLNSDGTLNIPAGVKEFTITTPVKADNTTEGEEKGKFTVG
GVEGNEVTVNDTSKQDEQPKI
(SEQ ID NO: 11)

TABLE 12
TLNSDGTLNIPAGVKEFTITTPVKADNTTEGEEKGKFTVGGVEGN
EVTVNDTSATPADPADKPTITSPDNDGKVTVEPGADNNKVEATFK
DEDGNDKTVVAEKGQDGNWTITNDDGTGATIEGGKVVIPADKVKD
GEPVNAKGTNDAGNSENADAVNAGTDPKDAGVDNTNGDGVVSAPV
SADEGTSIVTTVKLNNNNGNEKLPFSLPSGTGNGQLTADDFEQPT
FSNGVTLNSDGTLNIPAGVKEFTITTPVKADNTTEGEEKGKFTVG
GVEGNEVTVNDTSKQDEQPKI
(SEQ ID NO: 12)

TABLE 13
TLNSDGTLNIPAGVKEFTITTPVKADNTTEGEEKGKFTVGGVEGN
EVTVNDTSATPADPADKPTITSPDNDGKVTVEPGADNNKVEVTFK
DEDGNDKTVVAEKGQDGNWTITNDDGTGATIEGGKVVIPADKVKD
GEPVNAKGTNDAGNSENADAVNAGTDPKDAGVDNTNGDGVVSAPV
SADEGTNIVTTVKLNNNNGNEKLPFSLPSGTGNGQLTADDFEQPT
FSNGVTLNSDGTLNIPAGVKEFTITTPVKADNTTEGEEKGKFTVG
GVEGNEVTVNDTSKQDEQPKI
(SEQ ID NO: 13)

TABLE 14
TLNSDGTLNIPAGVKEFTITTPVKADNTTEGEEKGKFTVGGVEGN
EVTVNDTSATPADPADKPTITSPDNDGKVTVEPGADNNKVEVTFK
DEDGNDKTVVAEKGQDGNWTITNDDGTGATIEGGKVVIPADKVKD
GEPVNAKGTNDAGNSENADAVNAGTDPKDAGVDNTNGDGVVSAPV
SADEGTSIVTTVKLNNNNGNEKLPFSLPSGTGNGQLTADDFEQPT
FSNGVTLNSDGTLNIPAGVKEFTITTPVKADNTTEGEEKGKFTVG
GVEGNEVTVNDTSKQDEQPKI
(SEQ ID NO: 14)

TABLE 15
TLNQDGTLNVPAGVKEFTITTPVKADNTTEGEEKGKFTVGGVESN
EVTVNDTSATPADPADKPTITSPDNDGKVTVEPGADNNKVEVTFK
DEDGKDKTVVAEKGTDGNWTVTQDDGTGATIEGGKVVIPADKVKD
GEPVNAKGTNDAGNSENADAVNAGTDPKDAGVDNTNGDGVVSAPV
SADEGTNIVTTVKLNNNNGNEKLLFSLPNGTGNGQLTADDFEQPT
FSNGVTLNSDGTLNIPAGVKEFTITTPVKADNTTEGEEKGKFTVG
GVEGNEVTVNDTSKQDEQPKI
(SEQ ID NO: 15)

Among these polypeptides, all the NAIS61, the NAIS230, and the NAIS291 are a partial polypeptide of a protein (a 30 kDa protein named NAIS by the present inventors), which is obtained by deleting a portion considered to be immunogenic by the present inventors from the protein MP3 (100 kDa) obtained by deleting a portion considered to be cytotoxic from the outer membrane protein PnxIIIA (250 kDa) of Pasteurella pneumotropica.

Accordingly, as described in Example 1 below, a gene encoding the PnxIIIA protein is amplified by PCR from the genome of Pasteurella pneumotropica, the thus obtained gene product is used as a template to amplify a gene product of a region encoding from 1,393 to 1,683 amino acid residues of the PnxIIIA protein, the resultant is inserted into a pBAD vector having an araBAD promoter and a 6× histidine tag, and the resultant is incorporated into an E. coli strain in which the arab of an araBAD promoter is substituted by T7RNA polymerase. The polypeptide can be produced by culturing the thus transformed E. coli in LB medium.

Besides, the polypeptides containing NAIS61, NAIS230, and NAIS291 have known amino acid sequences, and hence can be produced by a general genetic engineering peptide synthesis method. Specifically, the polypeptide can be produced as follows: A DNA fragment encoding a desired amino acid sequence is prepared, the DNA fragment is incorporated into a vector, cells for producing the polypeptide are caused to incorporate the vector therein, only cells having the plasmid incorporated therein are selected, and the selected cells are grown to collect a polypeptide thus produced.

When the polypeptide is to be produced in E. coli, usable vectors are one having a target gene downstream of T71ac promoter, and using, as a host, an E. coli strain having T7RNA polymerase gene incorporated therein, or one having a target gene downstream of an araBAD promoter controlling metabolism of L-arabinose, and using, as a host, an E. coli strain lacking L-arabinose metabolic pathway or an E. coli strain in which the arab of an araBAD promoter is substituted by T7RNA polymerase.

A method for incorporating a vector into E. coli can be a heat shock method in which E. coli is treated with potassium chloride, and the temperature of the resultant is rapidly increased to cause the vector to be incorporated, or an electroporation method in which impurities are removed from E. coli, the resultant is suspended in a 10% glycerol solution, and a pulse of a high voltage is applied thereto to introduce a vector into the cells of the E. coli.

The cells having the plasmid incorporated therein can be selected by using a medium containing ampicillin, kanamycin, or chloramphenicol.

When an antigen is bound to the polypeptide, and the resultant is transmucosally administered, a specific antibody against the antigen can be remarkably produced. Here, an antibody against the polypeptide is little produced.

Accordingly, the polypeptide is useful as an adjuvant for producing antibodies against various antigens. Besides, a conjugate of the polypeptide with an antigen is useful as a polypeptide for producing an antibody against the antigen. Here, the various antigens include a pathogen or a substance produced by the pathogen, and a part of the pathogen or substance.

When a pathogen, or a substance produced by the pathogen, or a part of the pathogen or substance is bound to the polypeptide, and the resultant is transmucosally administered, a specific antibody against the pathogen can be remarkably produced. More specifically, when a pathogen, or a substance produced by the pathogen, or a part of the pathogen or substance is bound to the polypeptide, and the resultant is transmucosally administered, secretory IgA can be induced with a high antibody titer, and hence the invasion of the pathogen can be prevented on the mucosa. In addition, IgA and IgG can be induced with a high antibody titer also in the serum, and thus, an infectious disease caused by the pathogen or a substance produced by the pathogen having entered the body can be prevented. At this point, an antibody against the polypeptide is little produced. Furthermore, the polypeptide itself shows little cytotoxicity.

Accordingly, a conjugate of the polypeptide with a pathogen, a substance produced by the pathogen, or a part of the pathogen or substance is useful as a transmucosal vaccine.

Another embodiment of the present invention relates to a transmucosal vaccine containing, as an active ingredient, a conjugate of the polypeptide with a pathogen, a substance produced by the pathogen, or a part of the pathogen or substance.

Examples of the pathogen include viruses or bacterium which infect humans. Examples of the substance produced by the pathogen include proteins (including envelope proteins, membrane proteins, and spike proteins), toxins and the like produced by pathogens which infect humans.

Specific examples of the pathogen and the like include a pathogen selected from the group consisting of influenza virus, coronavirus, human papillomavirus, measles virus, tetanus toxin, and pertussis toxin, a substance produced by the pathogen, or a part of the pathogen or substance. Here, examples of the coronavirus include SARS-CoV, MERS-CoV, SARS-CoV-2, and common cold coronavirus.

Examples of the transmucosal vaccine of the present invention include those used as an inactivated vaccine, a VLP vaccine, and a recombinant protein vaccine.

An example of the pathogen, the substance produced by the pathogen, or a part of the pathogen or substance includes a part of the spike protein of coronavirus OC43, such as a polypeptide having an amino acid sequence shown in SEQ ID NO: 16 described below (OC43ag), and a polypeptide having an amino acid sequence having 95% or more identity to the amino acid sequence. The polypeptide having an amino acid sequence shown in SEQ ID NO: 16 (OC43ag) is an S2 subunit having high conservation among coronavirus spike proteins, and has 50% or more homology with the protein of SARS-CoV-2.

When a conjugate of the adjuvant of the present invention (the polypeptide) with OC43ag was nasally inoculated into a rabbit, not only a high antibody titer of an anti-coronavirus antibody was obtained but also a high antibody titer of an anti-SARS-CoV-2 spike protein was obtained.

Accordingly, the polypeptide having the amino acid sequence shown in SEQ ID NO: 16 (OC43ag) or the polypeptide having 95% or more identity to the amino acid sequence is useful as an antigen for producing a transmucosal vaccine for not only coronavirus but also SARS-CoV-2. Here, the polypeptide having an amino acid sequence having 95% or more identity to the amino acid sequence is preferably a polypeptide having an amino acid sequence having 97% or more identity to the amino acid sequence, more preferably a polypeptide having an amino acid sequence having 98% or more identity to the amino acid sequence, and furthermore preferably a polypeptide having an amino acid sequence having 99% or more identity to the amino acid sequence.

Examples of means for binding the adjuvant to an antigen (a pathogen, or a substance produced by the pathogen, or a part of the pathogen or substance) include means for directly binding the adjuvant to the antigen, means for producing, by genetic engineering, a polypeptide in which the adjuvant is bound to the antigen, and means for binding the adjuvant to the antigen via a linker.

Besides, a binding form between the adjuvant and the antigen may be a form in which the antigen is bound to either the N terminal side or the C terminal side of the adjuvant, and is preferably a form in which the antigen is bound to the C terminal side of the adjuvant.

The transmucosal vaccine of the present invention may contain the conjugate of the adjuvant (the polypeptide) with an antigen, and may further contain a pharmaceutically acceptable carrier. Examples of the pharmaceutically acceptable carrier include purified water, saline, various buffers, a pH adjuster, a stabilizer such as a sugar, and polyhydric alcohols.

The transmucosal vaccine of the present invention is a vaccine to be transmucosally administered. Among transmucosal administrations, nasal administration is preferred from the viewpoint of irritation and easiness of administration.

The form of the nasal administration may not be limited as long as the vaccine can be administered so as to attach from the mucosa of the nasal cavity to the mucosa of the upper respiratory, a cotton swab or the like may be used for direct administration, and a form in which a powder or spray droplets can reach the upper respiratory through the nasal cavity is preferred. Specific examples include an inhalation powder, an inhalation liquid, and an inhalation aerosol.

A dose of the transmucosal vaccine of the present invention varies depending on the type of a pathogen, and since the vaccine has strong adjuvant effect, the dose may be smaller than in single administration of a pathogen or the like. The frequency of administration varies depending on the type of a pathogen or the like, and can be appropriately selected.

### EXAMPLES

Now, the present invention will be described in more detail by way of examples, and it is noted that the present invention is not limited to these examples.

### Example 1

### (Production of NAIS291-1 (Polypeptide of SEQ ID NO: 11))

A gene encoding PnxIIIA protein was amplified by PCR from the genome of Pasteurella pneumotropica, the resultant gene product was used as a template to amplify a gene product of a region encoding from 1,393 to 1, 683 amino acid residues of PnxIIIA protein, the resultant was inserted into a pBAD vector having an araBAD promoter and a 6× histidine tag, and the resultant was incorporated into E. coli in which the arab of an araBAD promoter had been substituted by T7RNA polymerase. The thus transformed E. coli was cultured in LB medium for 3 hours, and then L-arabinose in a final concentration of 0.2% was added thereto, followed by culturing for another 3 hours. Bacterial cells thus obtained by culture were disrupted by sonication, and NAIS291-1 (polypeptide having an amino acid sequence shown in SEQ ID NO: 11) was separated and purified using magnetic beads for histidine tag purification.

Similarly, polypeptides respectively having amino acid sequences shown in SEQ ID NOS: 2, 3, 5 to 9, and 12 to 15 were obtained.

### Example 2

To the NAIS291-1 obtained in Example 1, a part of the spike protein of human coronavirus OC43 (OC43ag) was bound to produce NAIS291-OC43ag.

The vector obtained in Example 1 was used as a template to perform PCR using, as a starting point, the 3′ end of the gene encoding NAIS291-1 to linearize a NAIS291-1 expression vector. A part of an S2 subunit of a gene encoding a spike protein derived from human coronavirus OC43 was amplified by PCR, and the resultant was linked to the linear NAIS291-1 expression vector by PCR cloning. The resultant vector was incorporated into an E. coli strain in which the arab of an araBAD promoter had been substituted by T7RNA polymerase. The thus transformed E. coli was cultured in LB medium for 3 hours, and then L-arabinose in a final concentration of 0.2% was added thereto, followed by culturing for another 3 hours. Bacterial cells thus obtained by culture were disrupted by sonication, and NAIS291-OC43ag was separated and purified using magnetic beads for histidine tag purification.

The part of the spike protein of OC43 (OC43ag) used as the antigen had the following amino acid sequence:

TABLE 16
SGYTLAATSASLFPPWTAAAGVPFYLNVQYRINGLGVTMDVLSQN
QKLIANAFNNALHAIQQGFDATNSALVKIQAVVNANAEALNNLLQ
QLSNRFGAISASLQEILSRLDALEAEAQIDRLINGRLTALNAYVS
QQLSDSTLVKFSAAQAMEKVNECVKS
(SEQ ID NO: 16)

### Example 3

A solution of NAIS291-OC43ag in TBS (2 μM, 200 μl) was nasally inoculated into a rabbit three times with an interval of a week, and a serum antibody titer was measured 1 week after the third inoculation. Specifically, into a 96 well plate precoated with 100 μL of a Human coronavirus (HCoV-OC43) Spike S2 Protein solution (Sino Biological, China, 1 μg/ml) and treated with Blocking buffer (Pierce Protein-Free (PBS) Blocking Buffer), 100 μL of serially diluted rabbit serum was added to be reacted at 4° C. overnight, and the resultant was washed with 100 μL of 0.1% Tween 20 in PBS (PBST) 5 times. Subsequently, 100 ML of Anti-Rabbit IgG (H+L), HRP Conjugate (Promega, USA) or Anti-Rabbit IgA alpha chain (HRP) (Abcam, UK) diluted 3000 folds with Blocking buffer was added thereto to be reacted. The resultant was then washed with 100 μL of PBST 5 times, 100 μL of TMB microwell peroxidase Substrate (SeraCare Life Sciences, USA) was added thereto, the resultant was reacted at 25° C. for 10 minutes, and then an absorbance at 650 nm was measured. As a result, as illustrated in FIG. 2, as the anti-HCoV-CO43 spike protein antibody titer in serum, IgG showed an antibody titer higher than the upper limit of ELISA measurement. Thus, both IgG and IgA showed antibody titers sufficiently high for infection prevention.

### Example 4

To HCT-8 cells (2.0×10⁵ cells/200 μl), a control serum or a serum of a ribbit having been nasally inoculated with NAIS291-OC43ag (both in an amount of 100 μl) was added to be mixed, the resultant was then mixed with HCoV-OC43 (10,000 pfu/100 μl) for infecting the HCT-8 cells, and the resultant was cultured. Three weeks after starting the culture, cell degeneration was observed and virus copy number increased in the cells to which the control serum was added. In the cells to which the serum of a rabbit nasally inoculated with NAIS291-OC43ag was added, however, the HCT-8 cells were not degraded, and virus copy number, which was measured by real-time PCR by a method of Vijgen et al. (literature: Vijgen L., Keyaerts E., Moes E., Maes P., Duson G., Van Ranst M., Development of one-step, real-time, quantitative reverse transcriptase PCR assays for absolute quantitation of human coronaviruses OC43 and 229 E., J Clin Microbiol, 2005 43(11): 5452-5456), was equal to or lower than the detection lower limit.

### Example 5

OC43ag having an amino acid sequence shown in SEQ ID NO: 16 is an S2 subunit having high conservation among coronavirus spike proteins, and has 50% or more homology with the protein of SARS-CoV-2.

In a serum of a rabbit nasally inoculated with NAIS291-OC43ag, an antibody titer to SARS-CoV-2 spike protein S2 subunit was measured. Specifically, 100 μL of Recombinant SARS-CoV-2 S2 Subunit Protein with C-terminal His-tag, Transfected HEK293 Cell Culture Supernatant solution (RayBiotech Life, USA, 1 μg/ml) was used for precoating, and an antibody titer was measured in the same manner as in Example 3. As a result, an antibody titer of 1,024 or more was shown for IgG, and an antibody titer of 64 was shown for IgA, and thus, both the antibody titers were sufficiently high for infection protection.

### Example 6

To L929 cells (3.0×10³ cells/100 μl), BSA and NAIS291-1 respectively having been diluted with a medium to a final concentration of 10 μM, 1 μM, or 0.1 μM were added.

After culturing the resultant for 24 hours, cytotoxicity % was calculated with LDH cytotoxicity assay kit (Nacalai Tesque, Inc.). Four repeats were measured twice, and the average was obtained.

The cytotoxicity % was obtained by an expression, (abs of sample−abs of low control)/(abs of high control−abs of low control)×100.

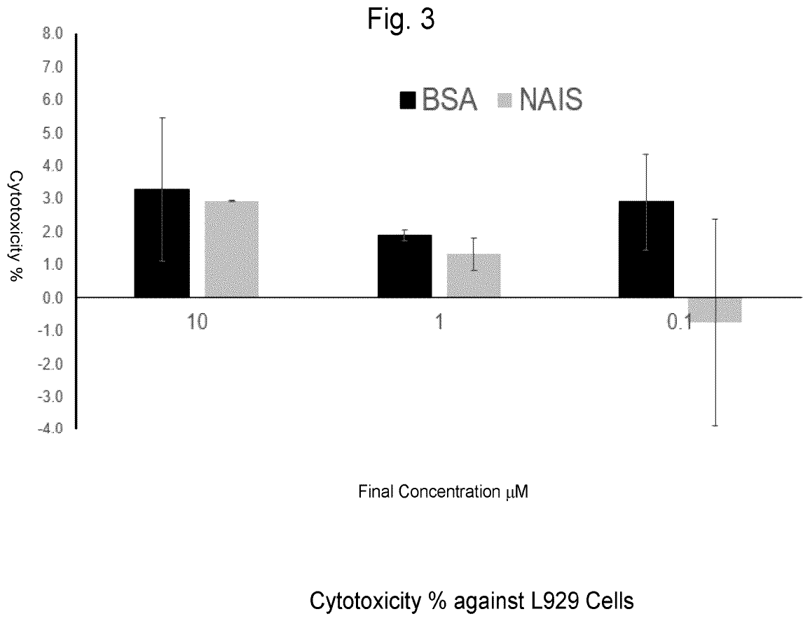

As a result, as illustrated in FIG. 3, NAIS291-1 (illustrated as NAIS in FIG. 3) had lower cytotoxicity than BSA used as the control.

### Example 7

Thioredoxin gene 6×HisTag (Trx-His: SEQ ID NO: 20) was used as an antigen to produce, in the same manner as in Example 2, a thioredoxin gene-NAIS291-1 conjugate (Trx-NAIS-His 291: SEQ ID NO: 17), a thioredoxin gene-NASI230-1 conjugate (Trx-NAIS-His 230: SEQ ID NO: 18), and a thioredoxin gene-NAIS61-2 conjugate (Trx-NAIS-His 61: SEQ ID NO: 19).

TABLE 17
Trx-NAIS-His 291
MGSDKIIHLTDDSFDTDVLKADGAILVDFWAHWCGPCKMIAPILD
EIADEYQGKLTVAKLNIDHNPGTAPKYGIRGIPTLLLFKNGEVAA
TKVGALSKGQLKEFLDANLAGSGSGDDDDKLGIITSLYKKAGSAA
ALFNFKKEPFTTLNSDGTLNIPAGVKEFTITTPVKADNTTEGEEK
GKFTVGGVEGNEVTVNDTSATPADPADKPTITSPDNDGKVTVEPG
ADNNKVEVTFKDEDGNDKTVVAEKGQDGNWTITNDDGTGATIEGG
KVVIPADKVKDGEPVNAKGTNDAGNSENADAVNAGTDPKDAGVNN
TNGDGVVSAPVSADEGTNIVTTVKLNNNNGNEKLPFSLPSGTGNG
QLTADDFEQPTFSNGVTLNSDGTLNIPAGVKEFTITTPVKADNTT
EGEEKGKFTVGGVEGNEVTVNDTSKQDEQPKIKGGRADSAFLYKV
VIKLEGKPIPNPLLGLDSTRTGHHHHHH
(SEQ ID NO: 17)

TABLE 18
Trx-NAIS-His 230
MGSDKIIHLTDDSFDTDVLKADGAILVDFWAHWCGPCKMIAPILD
EIADEYQGKLTVAKLNIDHNPGTAPKYGIRGIPTLLLFKNGEVAA
TKVGALSKGQLKEFLDANLAGSGSGDDDDKLGIITSLYKKAGSAA
ALFNFKKEPFTTLNSDGTLNIPAGVKEFTITTPVKADNTTEGEEK
GKFTVGGVEGNEVTVNDTSATPADPADKPTITSPDNDGKVTVEPG
ADNNKVEVTFKDEDGNDKTVVAEKGQDGNWTITNDDGTGATIEGG
KVVIPADKVKDGEPVNAKGTNDAGNSENADAVNAGTDPKDAGVNN
TNGDGVVSAPVSADEGTNIVTTVKLNNNNGNEKLPFSLPSGTGNG
QLTADDFEQPTFSNGVKGGRADSAFLYKVVIKLEGKPIPNPLLGL
DSTRTGHHHHHH
(SEQ ID NO: 18)

TABLE 19
Trx-NAIS-His 61
MGSDKIIHLTDDSFDTDVLKADGAILVDFWAHWCGPCKMIAPILD
EIADEYQGKLTVAKLNIDHNPGTAPKYGIRGIPTLLLFKNGEVAA
TKVGALSKGQLKEFLDANLAGSGSGDDDDKLGIITSLYKKAGSAA
ALFNFKKEPFTTLNSDGTLNIPAGVKEFTITTPVKADNTTEGEEK
GKFTVGGVEGNEVTVNDTSKQDEQPKIKGGRADSAFLYKVVIKLE
GKPIPNPLLGLDSTRTGHHHHHH
(SEQ ID NO: 19)

TABLE 20
Trx-His
MGSDKIIHLTDDSFDTDVLKADGAILVDFWAHWCGPCKMIAPILD
EIADEYQGKLTVAKLNIDHNPGTAPKYGIRGIPTLLLFKNGEVAA
TKVGALSKGQLKEFLDANLAGSGSGDDDDKGKPIPNPLLGLDSTR
TGHHHHHH
(SEQ ID NO: 20)

Each of solutions of these polypeptides in TBS (5 UM, 200 μl) was nasally inoculated into a rabbit 3 times with an interval of a week, and a serum antibody titer was measured 1 week after the third inoculation. Specifically, into a 96 well plate precoated with 100 μL of Trx-His solution (SEQ ID NO: 20, 1 g/ml) or NAIS291 solution (SEQ ID NO: 13, 1 μg/ml) and treated with Blocking buffer (Pierce Protein-Free (PBS) Blocking Buffer), 100 μL of serially diluted rabbit serum was added to be reacted at 4° C. overnight, and the resultant was then washed with 100 μL of PBST 5 times. Subsequently, 100 μL of Anti-Rabbit IgG (H+L) HRP Conjugate (Promega, USA) or Anti-Rabbit IgA alpha chain (HRP) (Abcam, UK) diluted 3000 folds with Blocking buffer was added thereto to be reacted, the resultant was then washed with 100 μL of PBST 5 times, 100 μL of TMB microwell peroxidase Substrate (SeraCare Life Sciences, USA) was added thereto, the resultant was reacted at 25° C. for 10 minutes, and then an absorbance at 650 nm was measured.

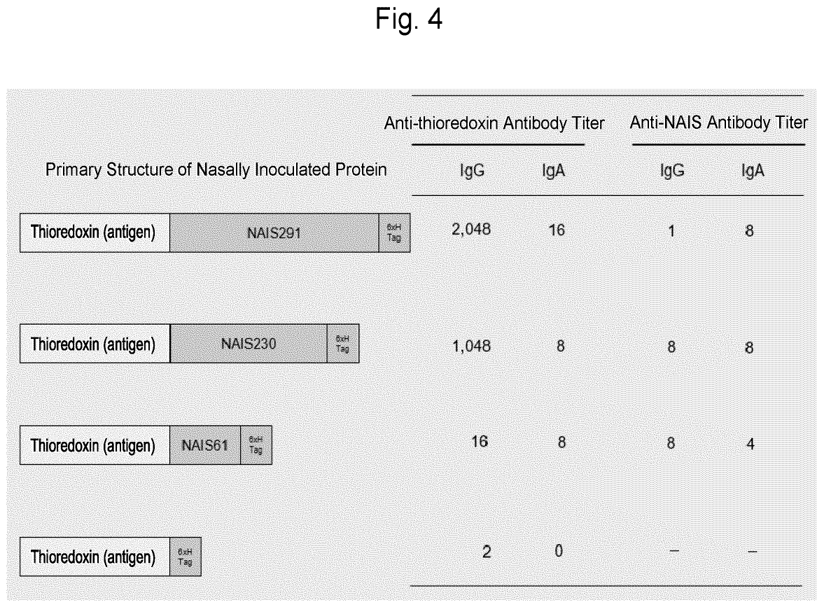

As a result, as illustrated in FIG. 4, the thioredoxin gene-NAIS61-2 conjugate (Trx-NAIS-His 61: SEQ ID NO: 19) provided an increased anti-thioredoxin antibody titer (IgG), and both the thioredoxin gene-NAIS291-1 conjugate (Trx-NAIS-His 291: SEQ ID NO: 17) and the thioredoxin gene-NAIS230-1 conjugate (Trx-NAIS-His 230: SEQ ID NO: 18) showed a very high anti-thioredoxin antibody titer (IgG).

On the other hand, the thioredoxin gene 6×HisTag (Trx-His: SEQ ID NO: 20) little induced the anti-thioredoxin antibody titer (IgG).

Besides, none of the polypeptides induced an anti-NAIS antibody titer.

### Example 8

NAIS230-1 and NAIS61-2 were crosslinked with influenza virus (Influenza A H1N1 (A/Puerto Rico/8/1934)) hemagglutinin 6×His (HA, Sino Biological, China) using N-Hydroxysuccinimide (Thermo Fisher Scientific, USA) in accordance with protocol provided by Thermo Fisher Scientific to produce NAIS230-HA and NAIS61-HA.

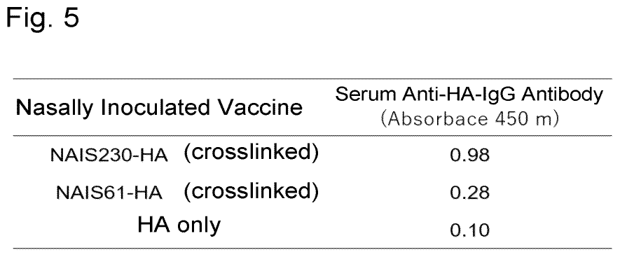

Each of solutions of NAIS230-HA, NAIS61-HA, and HA in TBS (0.1 μM, 20 μl) was nasally inoculated into a mouse 3 times with an interval of a week, and serum anti-HA-IgG antibody was measured by enzyme-linked immunosorbent assay 1 week after the third inoculation. Specifically, into a 96 well plate precoated with 100 μL of HA solution (1 μg/ml) and treated with Blocking buffer (Pierce Protein-Free T20), 100 μL of mouse serum was added to be reacted at 4° C. overnight, and the resultant was then washed with 200 μL of PBST 4 times. Subsequently, 100 μL of Goat anti-mouse IgG-Fc Fragment HRP Conjugated (Bethyl Laboratories, USA) diluted 5000 times with 10% Blocking buffer was added thereto to be reacted, the resultant was washed with 200 μL of PBST 7 times, 100 μL of TMB microwell peroxidase Substrate (SeraCare Life Sciences, USA) was added thereto, and the resultant was reacted at 25° C. for 10 minutes. Thereafter, 100 μL of 1 mol/L sulfuric acid was added thereto to stop the reaction, and then an absorbance at 450 nm was measured. As a result, as illustrated in FIG. 5, the absorbances (450 nm) of NAIS230-HA and NAIS61-HA were higher than that of single HA, which indicates that the anti-HA-IgG antibody had been induced.

1. An adjuvant, comprising, as an active ingredient:
a polypeptide consisting of an amino acid sequence show in SEQ ID NO: 1, 4, or 10.

2. An adjuvant, comprising, as an active ingredient:
a polypeptide consisting of an amino acid sequence shown in SEQ ID NO: 2, 3, 5, 6, 7, 8, 9, 11, 12, 13, 14, or 15.

3. An adjuvant, comprising, as an active ingredient:
a polypeptide consisting of an amino acid sequence shown in SEQ ID NO: 5, 6, 7, 8, 9, 11, 12, 13, 14, or 15.

4-8. (canceled)

9. A polypeptide comprising, as an active ingredient:
a conjugate of the adjuvant of claim 1 with an antigen, for producing an antibody against the antigen.

10. A transmucosal vaccine, comprising, as an active ingredient:
a conjugate of the adjuvant of claim 1 with a pathogen, a substance produced by the pathogen, or a part of the pathogen or substance.

11. The transmucosal vaccine of claim 10, wherein the pathogen, the substance produced by the pathogen, or the part of the pathogen or substance is an influenza virus, coronavirus, human papillomavirus, measles virus, tetanus toxin, and/or pertussis toxin, or a substance produced by the pathogen, or a part thereof.

12. A method of producing a transmucosal vaccine, the method comprising:
making a conjugate of the adjuvant of claim 1 with a pathogen, a substance produced by the pathogen, or a part of the pathogen or substance.

13. A method of conducting a vaccination, the method comprising:
administering to a subject an effective amount of a conjugate of the adjuvant of claim 1 with a pathogen, a substance produced by the pathogen, or a part of the pathogen or substance.

14. A transmucosal vaccine therapy, comprising:
transmucosally administering a conjugate of the adjuvant of claim 1 with a pathogen, a substance produced by the pathogen, or a part of the pathogen or substance.

15-17. (canceled)

18. A polypeptide comprising, as an active ingredient:
a conjugate of the adjuvant of claim 2 with an antigen, for producing an antibody against the antigen.

19. A transmucosal vaccine, comprising, as an active ingredient:
a conjugate of the adjuvant of claim 2 with a pathogen, a substance produced by the pathogen, or a part of the pathogen or substance.

20. The transmucosal vaccine of claim 19, wherein the pathogen, the substance produced by the pathogen, or the part of the pathogen or substance is an influenza virus, coronavirus, human papillomavirus, measles virus, tetanus toxin, and/or pertussis toxin, or a substance produced by the pathogen, or a part thereof.

21. A method of producing a transmucosal vaccine, the method comprising:
making a conjugate of the adjuvant of claim 2 with a pathogen, a substance produced by the pathogen, or a part of the pathogen or substance.

22. A method of conducting a vaccination, the method comprising:
administering to a subject an effective amount of a conjugate of the adjuvant of claim 2 with a pathogen, a substance produced by the pathogen, or a part of the pathogen or substance.

23. A transmucosal vaccine therapy, comprising:
transmucosally administering a conjugate of the adjuvant of claim 2 with a pathogen, a substance produced by the pathogen, or a part of the pathogen or substance.

24. A polypeptide comprising, as an active ingredient:
a conjugate of the adjuvant of claim 3 with an antigen, for producing an antibody against the antigen.

25. A transmucosal vaccine, comprising, as an active ingredient:
a conjugate of the adjuvant of claim 3 with a pathogen, a substance produced by the pathogen, or a part of the pathogen or substance.

26. The transmucosal vaccine of claim 25, wherein the pathogen, the substance produced by the pathogen, or the part of the pathogen or substance is an influenza virus, coronavirus, human papillomavirus, measles virus, tetanus toxin, and/or pertussis toxin, or a substance produced by the pathogen, or a part thereof.

27. A method of producing a transmucosal vaccine, the method comprising:
making a conjugate of the adjuvant of claim 3 with a pathogen, a substance produced by the pathogen, or a part of the pathogen or substance.

28. A method of conducting a vaccination, the method comprising:
administering to a subject an effective amount of a conjugate of the adjuvant of claim 3 with a pathogen, a substance produced by the pathogen, or a part of the pathogen or substance.

29. A transmucosal vaccine therapy, comprising:
transmucosally administering a conjugate of the adjuvant of claim 3 with a pathogen, a substance produced by the pathogen, or a part of the pathogen or substance.


SEQUENCE LISTING (20 sequences)

SEQ ID NO: 1 - protein, 61 aa, synthetic construct
  VARIANT 4: note = S or Q
  VARIANT 10: note = I or V
  VARIANT 44: note = G or S
  VARIANT 54: note = A or K
  VARIANT 55: note = T or Q
  VARIANT 56: note = P or D
  VARIANT 57: note = A or E
  VARIANT 58: note = D or Q
  VARIANT 60: note = A or K
  VARIANT 61: note = D or I
TLNXDGTLNXPAGVKEFTITTPVKADNTTEGEEKGKFTVGGVEXNEVTVNDTSXXXXXPXX

SEQ ID NO: 2 - protein, 61 aa, synthetic construct
TLNSDGTLNIPAGVKEFTITTPVKADNTTEGEEKGKFTVGGVEGNEVTVNDTSATPADPAD

SEQ ID NO: 3 - protein, 61 aa, synthetic construct
TLNSDGTLNIPAGVKEFTITTPVKADNTTEGEEKGKFTVGGVEGNEVTVNDTSKQDEQPKI

SEQ ID NO: 4 - protein, 230 aa, synthetic construct
  VARIANT 4: note = S or Q
  VARIANT 10: note = I or V
  VARIANT 44: note = G or S
  VARIANT 87: note = V or A
  VARIANT 95: note = N or K
  VARIANT 105: note = Q or T
  VARIANT 111: note = I or V
  VARIANT 113: note = N or Q
  VARIANT 168: note = D or N
  VARIANT 187: note = S or N
TLNXDGTLNXPAGVKEFTITTPVKADNTTEGEEKGKFTVGGVEXNEVTVNDTSATPADPADKPTITSPDNDGKVTVEPGADNNKVEXTFKDEDGXDKTVVAEKGXDGNWTXTXDDGTGATIEGGKVVIPADKVKDGEPVNAKGTNDAGNSENADAVNAGTDPKDAGVXNTNGDGVVSAPVSADEGTXIVTTVKLNNNNGNEKLPFSLPSGTGNGQLTADDFEQPTFSNGV

SEQ ID NO: 5 - protein, 230 aa, synthetic construct
TLNSDGTLNIPAGVKEFTITTPVKADNTTEGEEKGKFTVGGVEGNEVTVNDTSATPADPADKPTITSPDNDGKVTVEPGADNNKVEVTFKDEDGNDKTVVAEKGQDGNWTITNDDGTGATIEGGKVVIPADKVKDGEPVNAKGTNDAGNSENADAVNAGTDPKDAGVNNTNGDGVVSAPVSADEGTNIVTTVKLNNNNGNEKLPFSLPSGTGNGQLTADDFEQPTFSNGV

SEQ ID NO: 6 - protein, 230 aa, synthetic construct
TLNSDGTLNIPAGVKEFTITTPVKADNTTEGEEKGKFTVGGVEGNEVTVNDTSATPADPADKPTITSPDNDGKVTVEPGADNNKVEATFKDEDGNDKTVVAEKGQDGNWTITNDDGTGATIEGGKVVIPADKVKDGEPVNAKGTNDAGNSENADAVNAGTDPKDAGVDNTNGDGVVSAPVSADEGTSIVTTVKLNNNNGNEKLPFSLPSGTGNGQLTADDFEQPTFSNGV

SEQ ID NO: 7 - protein, 230 aa, synthetic construct
TLNSDGTLNIPAGVKEFTITTPVKADNTTEGEEKGKFTVGGVEGNEVTVNDTSATPADPADKPTITSPDNDGKVTVEPGADNNKVEVTFKDEDGNDKTVVAEKGQDGNWTITNDDGTGATIEGGKVVIPADKVKDGEPVNAKGTNDAGNSENADAVNAGTDPKDAGVDNTNGDGVVSAPVSADEGTNIVTTVKLNNNNGNEKLPFSLPSGTGNGQLTADDFEQPTFSNGV

SEQ ID NO: 8 - protein, 230 aa, synthetic construct
TLNSDGTLNIPAGVKEFTITTPVKADNTTEGEEKGKFTVGGVEGNEVTVNDTSATPADPADKPTITSPDNDGKVTVEPGADNNKVEVTFKDEDGNDKTVVAEKGQDGNWTITNDDGTGATIEGGKVVIPADKVKDGEPVNAKGTNDAGNSENADAVNAGTDPKDAGVDNTNGDGVVSAPVSADEGTSIVTTVKLNNNNGNEKLPFSLPSGTGNGQLTADDFEQPTFSNGV

SEQ ID NO: 9 - protein, 230 aa, synthetic construct
TLNQDGTLNVPAGVKEFTITTPVKADNTTEGEEKGKFTVGGVESNEVTVNDTSATPADPADKPTITSPDNDGKVTVEPGADNNKVEVTFKDEDGKDKTVVAEKGTDGNWTVTQDDGTGATIEGGKVVIPADKVKDGEPVNAKGTNDAGNSENADAVNAGTDPKDAGVDNTNGDGVVSAPVSADEGTNIVTTVKLNNNNGNEKLLFSLPNGTGNGQLTADDFEQPTFSNGV

SEQ ID NO: 10 - protein, 291 aa, synthetic construct
  VARIANT 4: note = S or Q
  VARIANT 10: note = I or V
  VARIANT 44: note = G or S
  VARIANT 87: note = V or A
  VARIANT 95: note = N or K
  VARIANT 105: note = Q or T
  VARIANT 111: note = I or V
  VARIANT 113: note = N or Q
  VARIANT 168: note = D or N
  VARIANT 187: note = S or N
TLNXDGTLNXPAGVKEFTITTPVKADNTTEGEEKGKFTVGGVEXNEVTVNDTSATPADPADKPTITSPDNDGKVTVEPGADNNKVEXTFKDEDGXDKTVVAEKGXDGNWTXTXDDGTGATIEGGKVVIPADKVKDGEPVNAKGTNDAGNSENADAVNAGTDPKDAGVXNTNGDGVVSAPVSADEGTXIVTTVKLNNNNGNEKLPFSLPSGTGNGQLTADDFEQPTFSNGVTLNSDGTLNIPAGVKEFTITTPVKADNTTEGEEKGKFTVGGVEGNEVTVNDTSKQDEQPKI

SEQ ID NO: 11 - protein, 291 aa, synthetic construct
TLNSDGTLNIPAGVKEFTITTPVKADNTTEGEEKGKFTVGGVEGNEVTVNDTSATPADPADKPTITSPDNDGKVTVEPGADNNKVEVTFKDEDGNDKTVVAEKGQDGNWTITNDDGTGATIEGGKVVIPADKVKDGEPVNAKGTNDAGNSENADAVNAGTDPKDAGVNNTNGDGVVSAPVSADEGTNIVTTVKLNNNNGNEKLPFSLPSGTGNGQLTADDFEQPTFSNGVTLNSDGTLNIPAGVKEFTITTPVKADNTTEGEEKGKFTVGGVEGNEVTVNDTSKQDEQPKI

SEQ ID NO: 12 - protein, 291 aa, synthetic construct
TLNSDGTLNIPAGVKEFTITTPVKADNTTEGEEKGKFTVGGVEGNEVTVNDTSATPADPADKPTITSPDNDGKVTVEPGADNNKVEATFKDEDGNDKTVVAEKGQDGNWTITNDDGTGATIEGGKVVIPADKVKDGEPVNAKGTNDAGNSENADAVNAGTDPKDAGVDNTNGDGVVSAPVSADEGTSIVTTVKLNNNNGNEKLPFSLPSGTGNGQLTADDFEQPTFSNGVTLNSDGTLNIPAGVKEFTITTPVKADNTTEGEEKGKFTVGGVEGNEVTVNDTSKQDEQPKI

SEQ ID NO: 13 - protein, 291 aa, synthetic construct
TLNSDGTLNIPAGVKEFTITTPVKADNTTEGEEKGKFTVGGVEGNEVTVNDTSATPADPADKPTITSPDNDGKVTVEPGADNNKVEVTFKDEDGNDKTVVAEKGQDGNWTITNDDGTGATIEGGKVVIPADKVKDGEPVNAKGTNDAGNSENADAVNAGTDPKDAGVDNTNGDGVVSAPVSADEGTNIVTTVKLNNNNGNEKLPFSLPSGTGNGQLTADDFEQPTFSNGVTLNSDGTLNIPAGVKEFTITTPVKADNTTEGEEKGKFTVGGVEGNEVTVNDTSKQDEQPKI

SEQ ID NO: 14 - protein, 291 aa, synthetic construct
TLNSDGTLNIPAGVKEFTITTPVKADNTTEGEEKGKFTVGGVEGNEVTVNDTSATPADPADKPTITSPDNDGKVTVEPGADNNKVEVTFKDEDGNDKTVVAEKGQDGNWTITNDDGTGATIEGGKVVIPADKVKDGEPVNAKGTNDAGNSENADAVNAGTDPKDAGVDNTNGDGVVSAPVSADEGTSIVTTVKLNNNNGNEKLPFSLPSGTGNGQLTADDFEQPTFSNGVTLNSDGTLNIPAGVKEFTITTPVKADNTTEGEEKGKFTVGGVEGNEVTVNDTSKQDEQPKI

SEQ ID NO: 15 - protein, 291 aa, synthetic construct
TLNQDGTLNVPAGVKEFTITTPVKADNTTEGEEKGKFTVGGVESNEVTVNDTSATPADPADKPTITSPDNDGKVTVEPGADNNKVEVTFKDEDGKDKTVVAEKGTDGNWTVTQDDGTGATIEGGKVVIPADKVKDGEPVNAKGTNDAGNSENADAVNAGTDPKDAGVDNTNGDGVVSAPVSADEGTNIVTTVKLNNNNGNEKLLFSLPNGTGNGQLTADDFEQPTFSNGVTLNSDGTLNIPAGVKEFTITTPVKADNTTEGEEKGKFTVGGVEGNEVTVNDTSKQDEQPKI

SEQ ID NO: 16 - protein, 161 aa, synthetic construct
SGYTLAATSASLFPPWTAAAGVPFYLNVQYRINGLGVTMDVLSQNQKLIANAFNNALHAIQQGFDATNSALVKIQAVVNANAEALNNLLQQLSNRFGAISASLQEILSRLDALEAEAQIDRLINGRLTALNAYVSQQLSDSTLVKFSAAQAMEKVNECVKS

SEQ ID NO: 17 - protein, 478 aa, synthetic construct
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKLGIITSLYKKAGSAAALFNFKKEPFTTLNSDGTLNIPAGVKEFTITTPVKADNTTEGEEKGKFTVGGVEGNEVTVNDTSATPADPADKPTITSPDNDGKVTVEPGADNNKVEVTFKDEDGNDKTVVAEKGQDGNWTITNDDGTGATIEGGKVVIPADKVKDGEPVNAKGTNDAGNSENADAVNAGTDPKDAGVNNTNGDGVVSAPVSADEGTNIVTTVKLNNNNGNEKLPFSLPSGTGNGQLTADDFEQPTFSNGVTLNSDGTLNIPAGVKEFTITTPVKADNTTEGEEKGKFTVGGVEGNEVTVNDTSKQDEQPKIKGGRADSAFLYKVVIKLEGKPIPNPLLGLDSTRTGHHHHHH

SEQ ID NO: 18 - protein, 417 aa, synthetic construct
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKLGIITSLYKKAGSAAALFNFKKEPFTTLNSDGTLNIPAGVKEFTITTPVKADNTTEGEEKGKFTVGGVEGNEVTVNDTSATPADPADKPTITSPDNDGKVTVEPGADNNKVEVTFKDEDGNDKTVVAEKGQDGNWTITNDDGTGATIEGGKVVIPADKVKDGEPVNAKGTNDAGNSENADAVNAGTDPKDAGVNNTNGDGVVSAPVSADEGTNIVTTVKLNNNNGNEKLPFSLPSGTGNGQLTADDFEQPTFSNGVKGGRADSAFLYKVVIKLEGKPIPNPLLGLDSTRTGHHHHHH

SEQ ID NO: 19 - protein, 248 aa, synthetic construct
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKLGIITSLYKKAGSAAALFNFKKEPFTTLNSDGTLNIPAGVKEFTITTPVKADNTTEGEEKGKFTVGGVEGNEVTVNDTSKQDEQPKIKGGRADSAFLYKVVIKLEGKPIPNPLLGLDSTRTGHHHHHH

SEQ ID NO: 20 - protein, 143 aa, synthetic construct
MGSDKIIHLTDDSFDTDVLKADGAILVDFWAHWCGPCKMIAPILDEIADEYQGKLTVAKLNIDHNPGTAPKYGIRGIPTLLLFKNGEVAATKVGALSKGQLKEFLDANLAGSGSGDDDDKGKPIPNPLLGLDSTRTGHHHHHH